(19) United States
(12) Patent Application Publication
(10) Pub. No.: US 2023/0157940 A1
(43) Pub. Date: May 25, 2023

(54) Oral Care Compositions and Methods for the Same

(71) Applicant: Colgate-Palmolive Company, New York, NY (US)

(72) Inventors: Melissa MARTINETTI, Bridgewater, NJ (US); David SURIANO, Edison, NJ (US)

(73) Assignee: Colgate-Palmolive Company, New York, NY (US)

(21) Appl. No.: 17/992,102

(22) Filed: Nov. 22, 2022

Related U.S. Application Data
(60) Provisional application No. 63/282,229, filed on Nov. 23, 2021.

(51) Int. Cl. A61K 8/73; A61Q 11/00; A61K 8/21; A61K 8/27

(52) U.S. Cl. CPC A61K 8/73; A61Q 11/00; A61K 8/21; A61K 8/27; A61K 2800/48

(57) ABSTRACT

Oral care compositions and methods for the same are disclosed. The oral care composition may include an orally acceptable vehicle, one or more fructans, and one or more thickening agents. The one or more thickening agents may be configured to prevent the crystallization of the one or more fructans in the orally acceptable vehicle. The method may include contacting the orally acceptable vehicle, the fructans, and the thickening agents with one another to thereby prevent the crystallization of the fructans.

### CROSS-REFERENCE TO RELATED APPLICATIONS

This application claims the benefit of U.S. Provisional Patent Application No. 63/282,229, filed on Nov. 23, 2021, which is incorporated herein by reference to the extent consistent with the present disclosure.

### BACKGROUND

At least 50 percent of the population suffer from chronic oral malodor or bad breath, and approximately half of these individuals experience a severe problem that creates personal discomfort and social embarrassment. The mouth air of those suffering from chronic oral malodor may often be infiltrated with compounds such as hydrogen sulfide, methyl mercaptan, and organic acids, which contribute to the oral malodor. These compounds are often produced by the proteolytic activity of microorganisms residing on the tongue and teeth. While those suffering from chronic oral malodor may often attempt to treat the condition with mints, chewing gum, compulsive brushing, and rinsing with mouthwashes, these often only mask the condition.

In view of the foregoing, oral care compositions incorporating inulin and/or zinc have been developed to mitigate or treat oral malodor. These conventional oral care compositions incorporating inulin and/or zinc, however, may often form precipitates or crystals at relatively low or freezing temperatures. The formation of crystals in the oral care composition is an undesirable phenomenon that is often unappealing to consumers in regions that experience relatively low or freezing temperatures.

What is needed, then, are improved oral care compositions incorporating inulin, zinc, or a combination thereof, and methods for the same.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing an oral care composition including an orally acceptable vehicle, one or more fructans, and one or more thickening agents, where the thickening agents may be configured to prevent the crystallization of the one or more fructans in the orally acceptable vehicle.

In at least one implementation, the one or more fructans may include one or more of inulin, levan, graminan, neo-inulin, neo-levan, or combinations thereof, preferably, the one or more fructants may include inulin.

In at least one implementation, the inulin may include a degree of polymerization of from 2 to about 60, preferably, a degree of polymerization of from 2 to less than 10.

In at least one implementation, the one or more fructans may be present in an amount effective to treat or prevent oral malodor, preferably, the one or more fructans may be present in an amount of from about 0.1 wt % to about 10 wt %, more preferably about 0.05 wt % to about 2 wt %, even more preferably about 0.05 wt % to about 1 wt %, based on the total weight of the oral care composition.

In at least one implementation, the one or more thickening agents may include a polymer configured to modify the viscosity of the oral care composition, preferably, the polymer may include a polysaccharide.

In at least one implementation, the polysaccharide may include one or more of a cellulose derivative, a polysaccharide gum, or a combination thereof, preferably, the polysaccharide may include the polysaccharide gum.

In at least one implementation, the polysaccharide gum may include one or more of xanthan gum, carrageenan gum, guar gum, succinoglucan gum, welan gum, gum Arabic, tragacanth gum, locust bean gum, or combinations thereof, preferably, the polysaccharide gum may include xanthan gum.

In at least one implementation, the thickening agents may be present in an amount effective to stabilize or facilitate the stabilization of the one or more fructans, preferably, the thickening agents may be present in an amount effective to stabilize or facilitate the stabilization of the one or more fructans upon exposure to an accelerated aging condition, more preferably, the accelerated aging condition may include exposure to temperatures at about 0° C. or less.

In at least one implementation, the thickening agents may be present in an amount of from about 0.1 wt % to about 10 wt %, more preferably about 0.05 wt % to about 2 wt %, even more preferably about 0.05 wt % to about 1 wt %, based on the total weight of the oral care composition.

In at least one implementation, the fructans and the thickening agents may be present in a weight ratio sufficient to stabilize or facilitate the stabilization of the fructans.

In at least one implementation, the weight ratio of the fructans to the thickening agents may be from about 0.1:1 to about 10:1, preferably about 0.5:1 to about 5:1, more preferably about 0.7:1 to about 2:1, even more preferably about 0.8:1 to about 1.2:1, or about 1:1.

In at least one implementation, the orally acceptable vehicle may include one or more of a humectant, a solvent, a pH modifying agent, a fluoride ion source, a zinc ion source, a desensitizing agent, a flavorant, or combinations thereof, preferably, the orally acceptable vehicle may include the humectant and the flavorant.

In at least one implementation, the humectant may include one or more of xylitol, glycerin, sorbitol, trimethylene glycol, or a combination thereof, preferably, the humectant may include a combination of xylitol, glycerin, trimethylene glycol, and sorbitol.

In at least one implementation, the flavorant may include menthol.

The foregoing and/or other aspects and utilities embodied in the present disclosure may be achieved by providing a method for preventing, inhibiting, or mitigating crystallization of the fructans of any of the oral care compositions disclosed herein. The method may include contacting the orally acceptable vehicle, the fructans, and the thickening agents with one another.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

The present inventors have surprisingly and unexpectedly discovered that the inclusion of a thickening agent prevents the formation of crystals or the crystallization of inulin in oral care compositions. The present inventors have also surprisingly and unexpectedly discovered that the inclusion of a thickening agent prevents the formation of crystals or the crystallization of inulin in oral care compositions when exposed to accelerated aging conditions, such as freezing temperatures. It was further surprisingly and unexpectedly discovered that oral care compositions including one or more fructans may be stabilized or may exhibit stability when combined with the thickening agent. The stability was maintained under and/or upon exposure to accelerated aging conditions.

Compositions disclosed herein may be or include an oral care product or an oral care composition thereof. For example, the composition may be an oral care product including the oral care composition and/or one or more additional ingredients/components. In another example, the composition may be the oral care composition of the oral care product. As used herein, the expression “oral care product” may refer to the final form which is sold to a consumer or administered to a user (e.g., patient). The oral care product may be a product that includes one or more chemical compounds or chemical compositions that may be applied to an oral cavity or a surface thereof to treat a condition (e.g., oral malodor), deliver an agent, improve the health of the user's oral cavity (e.g., vestibule, lips, jaws, palate, teeth, tongue, etc.), or a combination thereof.

The oral care product or the oral care composition thereof may be a liquid, a fluid, a gel, or a paste. Illustrative oral care products or compositions of the present disclosure may be or include, but are not limited to, a toothpaste (dentifrice), an oral rinse, a mouth rinse, a denture cleaner, a saliva substitute, a mouthwash, or any other oral care product intended to contact surfaces of the oral cavity. As used herein, the term or expression “toothpaste” may refer to a paste or gel dentifrice used with a toothbrush to clean and maintain the aesthetics and health of teeth and/or gums. As used herein, the terms or expressions “mouthwash,” “mouth rinse,” “oral rinse,” or the like, may refer to a liquid that contacts surfaces of the oral cavity or mouth passively or actively. In a preferred implementation, the oral care product or the composition thereof is a mouthwash.

The compositions disclosed herein may be or include oral care compositions including an orally acceptable vehicle or carrier, one or more fructans, one or more thickening agents, or a combination thereof. As further described herein, the one or more fructans may be capable of or configured to treat, prevent, or otherwise inhibit oral malodor, and the one or more thickening agents may be capable of or configured to stabilize or facilitate the stabilization of the one or more fructans contained in the oral care compositions.

Fructans include polymers of fructose molecules. For example, fructans may be or include oligosaccharides or polysaccharides including a sequence of anhydrofructose units optionally combined with one or more different saccharide residues of the fructose. Fructans may be linear or branched. Fructans may be products obtained directly from a plant or microbial source or else products with a chain length which has been modified (increased or reduced) by splitting, synthesis or hydrolysis, in particular of the enzymatic variety. Fructans generally have a degree of polymerization of from 2 to about 1,000 and preferably from 2 to about 60. For example, inulin has a degree of polymerization of from 2 to about 60. Fructans may be or include, or may be selected from, inulin, levan, graminan, neo-inulin, or neo-levan, or a combination thereof. In a preferred implementation, the fructan is or includes inulin.

The one or more fructans may be capable of or configured to treat, prevent, mitigate, or otherwise inhibit oral malodor. For example, the one or more fructans, such as inulin, may be capable of or configured to facilitate the growth or proliferation of bacteria associated with beneficial oral health. Illustrated bacteria associated with beneficial oral health may be or include, but is not limited to, Streptococcus salivarius, Streptococcus oralis, Streptococcus sanguinis, or the like, or a combination thereof.

Inulin is a fructan where fructosyl residues or fructose units thereof are linked by β-2-1 bonds. Inulin may be extracted from any suitable source, including, but not limited to, chicory, Agave, Agave spp., banana, plantain, Musaceae, burdock, Arctium lappa, camas Camassia spp., Cichorium intybus, coneflower, Echinacea spp., costus, Saussurea lappa, dandelion, Taraxacum officinale, elecampane, Inula helenium, garlic, allium sativum, globe artichoke, Cynara scolymus, Cynara cardunculus var. scolymus, Jerusalem artichoke, Helianthus tuberosus, jicama, Pachyrhizus erosus, Leopard's bane, Arnica montana, mugwort root, Artemisia vulgaris, onion, Allium cepa, wild yam, Dioscorea spp., yacón, Smallanthus sonchifolius, other similar tuberous plants, or the like, or a combination thereof. As noted above, inulin may have a degree of polymerization of from 2 to about 60. The inulin may be or include high-performance inulin, which may have a degree of polymerization of from 2 to less than 10.

The one or more fructans may be present in an amount effective to treat, prevent, or otherwise inhibit oral malodor. For example, the one or more fructans may be present in an amount of from about 0.01 wt % to about 10 wt %, based on the total weight of the composition. For example, the one or more fructans may be present in an amount of from about 0.01 wt % to about 10 wt %, preferably, about 0.05 wt % to about 5 wt %, more preferably about 0.05 wt % to about 1 wt %, even more preferably about 0.1 wt % to about 1 wt %, based on the total weight of the composition. In a preferred implementation, the one or more fructans include inulin in an amount of from about 0.01 wt % to about 10 wt %, about 0.01 wt % to about 0.5 wt %, about 0.01 wt % to about 0.1 wt %, preferably, about 0.05 wt % to about 5 wt %, more preferably about 0.05 wt % to about 2 wt %, even more preferably about 0.1 wt % to about 1 wt %, based on the total weight of the composition.

In at least one implementation, the oral care composition may include the one or more fructans in a relatively greater dose or amount to accommodate product dilution. For example, the oral care composition may include the one or more fructans in an amount of from greater than 10 wt % to about 20 wt %, about 30 wt %, about 50 wt %, or more, based on the total weight of the oral care composition. It should be appreciated that the dilution of the oral care composition may occur due to the differences between the oral care composition and the oral care product including the oral care composition.

The one or more thickening agents or viscosity modifying agents may be or include one or more polymers capable of or configured to modify (i.e., increase or decrease) the viscosity of the oral care composition. The one or more polymers may be or include water-dispersible or water-soluble hydrophilic colloids. Particularly, the one or more polymers may be or include polysaccharides. The one or more polymers or polysaccharides thereof may be unmodified, as isolated from their source materials, or may be modified as is well known in the polymer art, such as by acetylation, hydroxyalkylation, carboxyalkylation, hydroxyalkylation carboxyalkylation, cationic substitution, or a combination thereof. The one or more polymers may be or include natural and/or modified natural polymers and gums. Illustrative polysaccharides may be or include, but are not limited to, cellulose derivatives (e.g., carboxymethyl cellulose), gums or polysaccharide gums, or a combination thereof. Illustrative gums or polysaccharide gums may be or include, but are not limited to, xanthan gum, carrageenan gum, guar gum, succinoglucan gum, welan gum, gum Arabic, tragacanth gum, locust bean gum, or the like, or a combination thereof. In a preferred implementation, the one or more thickening agents include one or more polysaccharide gums selected from one or more of xanthan gum, carrageenan gum, guar gum, succinoglucan gum, welan gum, gum Arabic, tragacanth gum, locust bean gum, or the like, or a combination thereof, preferably, the one or more polysaccharide gums include xanthan gum.

The one or more thickening agents may be capable of or configured to stabilize or facilitate the stabilization of the one or more fructans contained in the oral care composition. For example, the one or more thickening agents may be capable of or configured to stabilize or facilitate the stabilization of the one or more fructans contained in the oral care composition. The one or more thickening agents may be capable of or configured to stabilize or facilitate the stabilization of the one or more fructans contained in the oral care composition upon exposure to one or more accelerated aging conditions (e.g., freezing temperatures). As used herein, the term or expression “stabilize” or “facilitate the stabilization of” may refer to inhibiting, preventing, or reducing the precipitation and/or crystallization of the one or more fructans in the oral care composition.

It should be appreciated that the precipitation and/or crystallization of the one or more fructans may result from the reaction, interaction, or otherwise contact between the one or more fructans and one or more of the remaining ingredients/components of the oral care composition. The formation of precipitates and/or crystals between the one or more fructans and the one or more remaining ingredients may be accelerated by or facilitated by exposure to one or more aging conditions, such as exposure to relatively low temperatures for a predetermined period of time (e.g., one hour, one day, one week, one month, etc.), a freeze-thaw condition, or a combination thereof. For example, the formation of precipitates and/or crystals between the one or more fructans and the one or more remaining ingredients may be accelerated by or facilitated by exposure to freezing temperature (e.g., less than or equal to about 0° C.) for a period of at least one minute, at least one hour, at least one day, at least one week, or at least one month, or more. The freezing temperature may include temperatures at about 0° C. or below. For example, the freezing temperature may be less than or equal to 0° C., less than or equal to −5° C., less than or equal to −10° C., or less than or equal to −20° C. It may also be appreciated that the one or more fructans may precipitate and/or crystalize alone upon exposure to the aging conditions disclosed above. For example, the formation of precipitates and/or crystals may not be from interactions or contact between the one or more fructans and one or more of the remaining components.

The one or more thickening agents may be present in an amount effective to stabilize or facilitate the stabilization of the one or more fructans contained in the oral care composition. For example, any one or more of the thickening agents may be present in an amount of from about 0.01 wt % to about 40 wt %, based on the total weight of the composition. For example, any one or more of the thickening agents may be present in an amount of from about 0.01 wt % to about 40 wt %, preferably, about 0.05 wt % to about 30 wt %, more preferably about 0.05 wt % to about 15 wt %, even more preferably about 0.1 wt % to about 1 wt %, based on the total weight of the composition. In a preferred implementation, the one or more thickening agents include at least xanthan gum in an amount of from about 0.01 wt % to about 2 wt %, about 0.01 wt % to about 1 wt %, about 0.01 wt % to about 0.5 wt %, preferably, about 0.05 wt % to about 5 wt %, more preferably, about 0.05 wt % to about 2 wt %, about 0.05 wt % to about 1 wt %, even more preferably about 0.1 wt % to about 1 wt %, based on the total weight of the composition.

The one or more fructans and the one or more thickening agents may be present in a weight ratio sufficient or effective to stabilize or facilitate the stabilization of the one or more fructans contained in the oral care composition. For example, the one or more fructans and the one or more thickening agents may be present in a weight ratio of from about 0.1:1 to about 10:1. For example, the one or more fructans and the one or more thickening agents may be present in a weight ratio of from about 0.1:1, about 0.2:1, about 0.3:1, about 0.4:1, about 0.5:1, about 0.6:1, about 0.7:1, about 0.8:1, about 0.9:1, or about 1:1 to about 1.1:1, about 1.2:1, about 1.3:1, about 1.4:1, about 1.5:1, about 1.6:1, about 1.7:1, about 1.8:1, about 1.9:1, about 2:1, about 3:1, about 4:1, about 5:1, about 6:1, about 7:1, about 8:1, about 9:1, or about 10:1. In a preferred implementation, the weight ratio of the fructans to the thickening agents may be from about 0.1:1 to about 10:1, preferably about 0.5:1 to about 5:1, more preferably about 0.7:1 to about 2:1, even more preferably about 0.8:1 to about 1.2:1, or about 1:1.

As discussed above, the composition may include the orally acceptable vehicle or carrier, the one or more fructans, the one or more thickening agents, or a combination thereof. As used herein, the expression “orally acceptable vehicle” or “carrier” may refer to a suitable vehicle, ingredient, or combination of ingredients, which may be utilized to dissolve, disperse, suspend, hold, mobilize, or otherwise contain the one or more fructans and/or the one or more thickening agents. In a preferred implementation, the composition includes the one or more fructans and the one or more thickening agents dispersed or otherwise contained in the orally acceptable vehicle or carrier. The orally acceptable vehicle may include one or more humectants, one or more solvents, one or more pH modifying agents, one or more fluoride ion sources, one or more zinc ion sources, one or more desensitizing agents, one or more flavorants or flavoring agents, one or more additional ingredients, or a combination thereof.

As used herein, the term or expression “humectant” may refer to a substance having affinity for water with stabilizing action on the water content of a material. The one or more humectants may include polyols, such as edible polyhydric alcohols (e.g., sugar alcohols). Illustrative humectants may be or include, but are not limited to, glycerin, butylene glycol, propylene glycol, sorbitol, xylitol, polyethylene glycol, 1,3-propanediol or trimethylene glycol, or the like, or a combination thereof. In a preferred implementation, the humectants of the oral care composition include xylitol, glycerin, sorbitol, trimethylene glycol, or a combination thereof. For example, the humectants include a combination of xylitol, glycerin, trimethylene glycol, and sorbitol.

The one or more humectants may be present in an amount of from 5 weight % to about 60 weight %, based on the total weight of the composition. For example, any one or more of the humectants may be present in an amount of from about 5 weight %, about 10 weight %, about 15 weight %, or about 20 weight % to about 25 weight %, about 30 weight %, about 35 weight %, about 40 weight %, about 45 weight %, about 50 weight %, about 55 weight %, or about 60 weight %, based on the total weight of the composition. In another example, any one or more of the humectants may be present in an amount of from about 5 weight % to about 60 weight %, about 10 weight % to about 55 weight %, about 15 weight % to about 50 weight %, about 20 weight % to about 30 weight %, or about 20 weight % to about 25 weight %, based on the total weight of the composition. In a preferred implementation, the humectants may be present in an amount of about 15 weight % to about 25 weight %, preferably about 20 weight % to about 25 weight %, more preferably about 20 weight % to about 23 weight %, or about 21 weight %, based on the total weight of the composition.

As noted above, in the preferred implementation, the humectants include xylitol, glycerin, trimethylene glycol, and sorbitol. The xylitol may be present in an amount of from about 4 wt % to about 12 wt %, preferably about 6 wt % to about 10 wt %, more preferably about 8 wt %, based on the total weight of the composition. The glycerin may be present in an amount of from about 4 wt % to about 12 wt %, preferably about 6 wt % to about 10 wt %, more preferably about 8 wt %, based on the total weight of the composition. The sorbitol may be present in an amount of from about 2 wt % to about 8 wt %, preferably about 4 wt % to about 6 wt %, more preferably about 5 wt %, based on the total weight of the composition. The trimethylene glycol may be present in an amount of from about 1 wt % to about 5 wt %, preferably, about 2 wt % to about 4 wt %, more preferably about 3 wt %, based on the total weight of the composition.

The one or more solvents of the orally acceptable vehicle may be or include any suitable solvent compatible with the remaining components of the oral care product or the oral care composition thereof. Illustrative solvents of the orally acceptable vehicle may be or include, but are not limited to, water, such as deionized or demineralized water, ethanol, or the like, or a combination thereof. In a preferred implementation, the orally acceptable vehicle includes water, more preferably demineralized water. Water may make up the balance of the oral care composition or the orally acceptable vehicle thereof. Water may be present in an amount of at least 60 weight %, at least 62 weight %, at least 64 weight %, at least 66 weight %, at least 68 weight %, at least 70 weight %, at least 72 weight %, at least 74 weight %, at least 76 weight %, at least 78 weight %, at least 80 weight %, at least 82 weight %, at least 84 weight %, at least 86 weight %, at least 88 weight %, at least 90 weight %, at least 92 weight %, at least 94 weight %, at least 96 weight %, at least 98 weight %, at least 99 weight %, or more, based on the total weight of the oral care composition or the orally acceptable vehicle thereof.

The one or more pH modifying agents may be or include one or more bases or basifying agents, one or more acids or acidifying agents, one or more buffers or buffering agents, or a combination thereof. The one or more pH modifying agents may be capable of or configured to provide the oral care product or the oral care composition thereof a pH of from 2 to 10, from 4 to 9, from 5 to 8, from 5 to 7, from 7 to 8, from 6 to 7, or about 7. Illustrative pH modifying agent are known in the art and may include, but are not limited to, carboxylic, phosphoric, and sulfonic acids, acid salts (e.g., monosodium citrate, disodium citrate, monosodium malate, etc.), alkali metal hydroxides, such as sodium hydroxide, carbonates, such as sodium carbonate, bicarbonates, sesquicarbonates, borates, silicates, phosphates (e.g., monosodium phosphate, trisodium phosphate, pyrophosphate salts, etc.), or the like, or a combination thereof. It should be appreciated that any one or more of the pH modifying agents may be present in an amount effective to maintain the composition in an orally acceptable pH range.

A wide variety of fluoride ion-yielding materials may be employed as sources of soluble fluoride. Examples of suitable fluoride ion-yielding materials may be found in U.S. Pat. No. 3,535,421 to Briner et al., U.S. Pat. No. 4,885,155 to Parran, Jr. et al., and U.S. Pat. No. 3,678,154 to Widder et al., the disclosures of which are incorporated herein by reference. Illustrative fluoride ion sources include, but are not limited to, stannous fluoride, sodium fluoride, potassium fluoride, sodium monofluorophosphate, sodium fluorosilicate, ammonium fluorosilicate, amine fluoride, ammonium fluoride, or a combination thereof. In a preferred embodiment, the fluoride ion source includes sodium fluoride. The amount of the fluoride ion source in the oral care composition may be greater than 0 wt % and less than 0.080 wt %, less than 0.07 wt %, less than 0.060 wt %, less than 0.050 wt %, less than 0.040 wt %, less than 0.030 wt %, or less than 0.025 wt %, based on the total weight of the oral care composition or the orally acceptable vehicle thereof. The fluoride ion source may also be present in an amount effective to provide a total of about 100 to about 20,000 ppm, about 200 to about 5,000 ppm, or about 500 to about 2,500 ppm, fluoride ions.

The one or more zinc ion sources may be capable of or configured to provide one or more zinc ions. The zinc ion source may be or include a chemical compound, a complex, or a zinc salt capable of or configured to provide the zinc ions. Illustrative zinc ion sources may be or include, but are not limited to, zinc sulfate, zinc chloride, zinc acetate, zinc phenolsulfonate, zinc borate, zinc bromide, zinc nitrate, zinc glycerophosphate, zinc benzoate, zinc carbonate, zinc citrate (e.g., zinc citrate trihydrate), zinc hexafluorosilicate, zinc lactate trihydrate, zinc oxide, zinc peroxide, zinc salicylate, zinc silicate, zinc stannate, zinc tannate, zinc tartrate, zinc titanate, zinc tetrafluoroborate, or a combination thereof. In a preferred implementation, the zinc ion source includes zinc citrate trihydrate. Any one or more of the zinc ion sources may be present in an amount of from about 0.01 wt % to about 5 wt %, preferably from about 0.05 wt % to about 1 wt %, more preferably about 0.1 wt % to about 0.5 wt %, even more preferably about 0.13 wt % to about 0.15 wt %, based on the total weight of the oral care composition or the orally acceptable vehicle thereof.

The one or more desensitizing agents may be or include, but are not limited to, potassium tartrate, potassium bicarbonate, potassium oxalate, potassium nitrate, strontium salts, or a combination thereof.

The one or more flavorants or flavoring agents may be or include, but are not limited to, sweeteners, flavoring oils or essential oils, sucrose, sucralose, dextrose, polydextrose, dextrin, mannose, xylose, ribose, fructose, levulose, galactose, corn syrup, including high fructose corn syrup and corn syrup solids, partially hydrolyzed starch, lactose, maltose, xylitol, stevia, sodium cyclamate, perillartine, aspartame, liquorice, hydrogenated starch hydrolysate, sorbitol, mannitol, maltitol, isomalt, aspartame, neotame, saccharin and salts thereof (e.g., sodium saccharin), dipeptide-based intense sweeteners, cyclamates, dihydrochalcones saccharin or a salt thereof, or a combination thereof. Examples of the essential oils include oils of spearmint, peppermint, wintergreen, menthol, sassafras, clove, sage, eucalyptus, marjoram, cinnamon, lemon, lime, grapefruit, and orange. Any one or more of the flavorants or flavoring agents may be present in an amount of from greater than 0 wt % to less than or equal to 1 wt %, less than or equal to 0.8 wt %, less than or equal to 0.7 wt %, less than or equal to 0.6 wt %, less than or equal to 0.5 wt %, less than or equal to 0.4 wt %, less than or equal to 0.35 wt %, less than or equal to 0.30 wt %, less than or equal to 0.25 wt %, less than or equal to 0.20 wt %, less than or equal to 0.1 wt %, or less than or equal to 0.05 wt %. In a preferred implementation, the flavoring agents include one or more essential oils, menthol, or combinations thereof. In an even more preferred implementation, the flavoring agents includes at least menthol. The menthol is preferably present in an amount of from about 0.01 wt % to about 0.05 wt %, preferably about 0.02 wt %.

It should be appreciated by one having ordinary skill in the art, that the oral care products and/or the oral care composition thereof may include other additional ingredients/components. For example, the oral care products and/or the oral care composition thereof may include any one or more of the following: anti-caries agents, diluents, surface active agents or surfactants, mouth feel agents, sweetening agents, colorants or coloring agents, preservatives, antifoam agents (e.g., benzoic acid, sulfuric acid, glyceryl monostearate, etc.), or the like, or a combination thereof. It should further be appreciated by one having ordinary skill in the art that while general attributes of each of the above categories of materials may differ, there may be some common attributes and any given material may serve multiple purposes within two or more of such categories of materials.

The present disclosure may provide methods for preventing, inhibiting, reducing, or otherwise mitigating precipitation and/or crystallization of one or more fructans in an oral care product or the oral care composition thereof. The method may include contacting the contacting an orally acceptable vehicle or carrier, the one or more fructans, and one or more thickening agents with one another. The one or more fructans may be or include inulin. The one or more thickening agents may be or include one or more gums or polysaccharide gums, such as xanthan gum. The method may also include preventing, inhibiting, reducing, or otherwise mitigating precipitation and/or crystallization of the one or more fructans under accelerated aging conditions. The accelerated aging conditions may include exposure to temperatures of less than or equal to about 0° C. For example, the accelerated aging conditions may include exposure of the oral care product or the oral care composition thereof to temperatures of less than or equal to 0° C., less than or equal to −5° C., less than or equal to −10° C., or less than or equal to −20° C. The method may include preventing, inhibiting, reducing, or otherwise mitigating precipitation and/or crystallization of the one or more fructans under accelerated aging conditions for at least one minute, at least one hour, at least one day, at least one week, at least one month, or more.

The present disclosure may provide methods for preparing oral care products or oral care compositions thereof, where the oral care product or the oral care composition exhibits increased stability under accelerated aging conditions. The method may include contacting an orally acceptable carrier, one or more fructans, and one or more thickening agents with one another. The one or more fructans may include inulin, the one or more thickening agents may include polysaccharide gums selected from one or more of xanthan gum, carrageenan gum, guar gum, succinoglucan gum, welan gum, gum Arabic, tragacanth gum, locust bean gum, or the like, or a combination thereof.

The present disclosure also provides methods for mitigating oral malodor of a user's oral cavity. The method may include contacting any of the oral care products or the oral care compositions thereof with a surface of the oral cavity of the user. The oral care product and/or the oral care composition thereof may be applied and/or contacted with the surfaces of the oral cavity at predetermined intervals. For example, a daily basis, at least once a day for multiple days, or alternatively every other day. In another example, the oral care product and/or the oral care composition thereof may be applied and/or contacted with the surfaces of the oral cavity at least once a day, at least once every two days, at least once every three days, at least once every five days, at least once a week, at least once every two weeks, or at least once a month. The oral care product and/or the oral care composition thereof may be utilized for up to 2 weeks, up to 3 weeks, up to 4 weeks, up to 6 weeks, up to 8 weeks, or greater.

The following numbered paragraphs disclose one or more exemplary variations of the subject matter of the application:

1. An oral care composition, comprising: an orally acceptable vehicle; one or more fructans; and one or more thickening agents.

2. The oral care composition of paragraph 1, wherein the thickening agents are configured to prevent precipitation and/or crystallization of the one or more fructans in the orally acceptable vehicle.

3. The oral care composition of paragraph 1 or 2, wherein the one or more fructans comprise one or more of inulin, levan, graminan, neo-inulin, neo-levan, or combinations thereof, preferably, the one or more fructants comprise inulin.

4. The oral care composition of paragraph 3, wherein the inulin comprises a degree of polymerization of from 2 to about 60, preferably, a degree of polymerization of from 2 to less than 10.

5. The oral care composition of any of paragraphs 1 to 4, wherein the one or more fructans are present in an amount effective to treat or prevent oral malodor, preferably, the one or more fructans are present in an amount of from about 0.1 wt % to about 10 wt %, more preferably about 0.05 wt % to about 2 wt %, even more preferably about 0.05 wt % to about 1 wt %, based on the total weight of the oral care composition.

6. The oral care composition of any of paragraphs 1 to 5, wherein the one or more thickening agents comprises a polymer configured to modify the viscosity of the oral care composition, preferably, the polymer comprises a polysaccharide.

7. The oral care composition of paragraph 6, wherein the polysaccharide comprises one or more of a cellulose derivative, a polysaccharide gum, or a combination thereof, preferably, the polysaccharide comprises the polysaccharide gum.

8. The oral care composition of claim 7, wherein the polysaccharide gum comprises one or more of xanthan gum, carrageenan gum, guar gum, succinoglucan gum, welan gum, gum Arabic, tragacanth gum, locust bean gum, or combinations thereof, preferably, the polysaccharide gum comprises xanthan gum.

9. The oral care composition of any of paragraphs 1 to 8, wherein the thickening agents are present in an amount effective to stabilize or facilitate the stabilization of the one or more fructans, preferably, the thickening agents are present in an amount effective to stabilize or facilitate the stabilization of the one or more fructans upon exposure to an accelerated aging condition, more preferably, the accelerated aging condition comprises exposure to temperatures at about 0° C. or less.

10. The oral care composition of any of paragraphs 1 to 9, wherein the thickening agents are present in an amount of from about 0.1 wt % to about 10 wt %, more preferably about 0.05 wt % to about 2 wt %, even more preferably about 0.05 wt % to about 1 wt %, based on the total weight of the oral care composition.

11. The oral care composition of any of paragraphs 1 to 10, wherein the fructans and the thickening agents are present in a weight ratio sufficient to stabilize or facilitate the stabilization of the fructans.

12. The oral care composition of paragraph 11, wherein the weight ratio of the fructans to the thickening agents is from about 0.1:1 to about 10:1, preferably about 0.5:1 to about 5:1, more preferably about 0.7:1 to about 2:1, even more preferably about 0.8:1 to about 1.2:1, or about 1:1.

13. The oral care composition of any of paragraphs 1 to 12, wherein the orally acceptable vehicle comprises one or more of a humectant, a solvent, a pH modifying agent, a fluoride ion source, a zinc ion source, a desensitizing agent, a flavorant, or combinations thereof, preferably, the orally acceptable vehicle comprises the humectant and the flavorant.

14. The oral care composition of paragraph 13, wherein the humectant comprises one or more of xylitol, glycerin, sorbitol, trimethylene glycol, or a combination thereof, preferably, the humectant comprises a combination of xylitol, glycerin, trimethylene glycol, and sorbitol.

15. The oral care composition of paragraph 14, wherein the flavorant comprises menthol.

16. A method for preventing, inhibiting, or mitigating crystallization and/or precipitation of the fructans of the oral care composition of any of paragraphs 1 to 15, the method comprising contacting the orally acceptable vehicle, the fructans, and the thickening agents with one another.

All ingredients for use in the compositions described herein should be orally acceptable. As used herein, “orally acceptable” may refer any ingredient that is present in a composition as described in an amount and form which does not render the composition unsafe for use in the oral cavity.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

The efficacy of thickening agents in preventing or inhibiting the formation of precipitates and/or crystals in oral care compositions incorporating inulin and zinc was evaluated. Particularly, the efficacy of xanthan gum, a representative thickening agent, in preventing or inhibiting the formation of crystals in the oral care compositions when exposed to freezing conditions was evaluated. To evaluate the efficacy of xanthan gum, a control oral care composition (1) and a test oral care composition (2) were prepared by combining the ingredients/components according to Table 1. As indicated in Table 1, the control oral care composition (1) excluded xanthan gum, and the test oral care composition (2) included xanthan gum. It should be appreciated that each of the remaining components of the control (1) and test (2) oral care compositions were either the same or substantially the same. As such, any changes or differences observed between the control (1) and test (2) oral care compositions may be attributed to the inclusion or exclusion of the thickening agent.

TABLE 1
Control (1) and Test (2) Oral Care Compositions

| | CONTROL | TEST |
|---|---|---|
| INGREDIENT/ | (1) | (2) |
| COMPONENT | (wt %) | (wt %) |
| Water | 74.465 | 74.615 |
| Humectant | 24.5 | 24.5 |
| Flavor | 0.6 | 0.6 |
| Preservative | 0.15 | 0.2 |
| Zinc Salt | 0.135 | 0.135 |
| Xanthan Gum | — | 0.1 |
| Inulin | 0.1 | 0.1 |
| pH Modifying Agent | 0.03 | 0.03 |
| Menthol | 0.02 | 0.02 |
| Total | 100.0 | 100.0 |

Each of the control (1) and test (2) oral care compositions was exposed to freezing conditions to evaluate the efficacy of the thickening agent. Particularly, the oral care compositions (1) and (2) were maintained at a temperature of about −10° C. for a month, and subsequently evaluated visually. It was surprisingly and unexpected discovered that the inclusion of a thickening agent, namely, xanthan gum, prevented the formation of crystals in the test oral care composition (2). The control oral care composition (2), which excluded the thickening agent, however, exhibited the formation of needle-like crystals. Without being bound by theory, it is believed that the crystal formation is the result of interactions between the inulin and one or more components of the oral care composition, such as, a flavorant (i.e., methanol). It is further believed that the inclusion of the thickening agent resulted in increased viscosity that discouraged the formation of the crystals.

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

What is claimed is:

1. An oral care composition, comprising:
an orally acceptable vehicle;
one or more fructans; and
one or more thickening agents, wherein the thickening agents are configured to prevent crystallization of the one or more fructans in the orally acceptable vehicle.

2. The oral care composition of claim 1, wherein the one or more fructans comprise one or more of inulin, levan, graminan, neo-inulin, neo-levan, or combinations thereof.

3. The oral care composition of claim 2, wherein the one or more fructans comprise inulin, and wherein the inulin comprises a degree of polymerization of from 2 to about 60.

4. The oral care composition of claim 1, wherein the one or more fructans are present in an amount effective to treat or prevent oral malodor.

5. The oral care composition of claim 1, wherein the one or more thickening agents comprises a polymer configured to modify the viscosity of the oral care composition.

6. The oral care composition of claim 5, wherein the polymer comprises a polysaccharide, wherein the polysaccharide comprises one or more of a cellulose derivative, a polysaccharide gum, or a combination thereof.

7. The oral care composition of claim 6, wherein the polysaccharide gum comprises one or more of xanthan gum, carrageenan gum, guar gum, succinoglucan gum, welan gum, gum Arabic, tragacanth gum, locust bean gum, or combinations thereof.

8. The oral care composition of claim 1, wherein the thickening agents are present in an amount effective to stabilize or facilitate the stabilization of the one or more fructans, preferably, the thickening agents are present in an amount effective to stabilize or facilitate the stabilization of the one or more fructans upon exposure to an accelerated aging condition, more preferably, the accelerated aging condition comprises exposure to temperatures at about 0° C. or less.

9. The oral care composition of claim 1, wherein the thickening agents are present in an amount of from about 0.1 wt % to about 10 wt %, based on the total weight of the oral care composition.

10. The oral care composition of claim 1, wherein the fructans and the thickening agents are present in a weight ratio sufficient to stabilize or facilitate the stabilization of the fructans.

11. The oral care composition of claim 10, wherein the weight ratio of the fructans to the thickening agents is from about 0.1:1 to about 10:1.

12. The oral care composition of claim 1, wherein the orally acceptable vehicle comprises one or more of a humectant, a solvent, a pH modifying agent, a fluoride ion source, a zinc ion source, a desensitizing agent, a flavorant, or combinations thereof.

13. The oral care composition of claim 12, wherein the humectant comprises one or more of xylitol, glycerin, sorbitol, trimethylene glycol, or a combination thereof.

14. The oral care composition of claim 12, wherein the flavorant comprises menthol.

15. A method for preventing, inhibiting, or mitigating crystallization of the fructans of the oral care composition of claim 1, the method comprising contacting the orally acceptable vehicle, the fructans, and the thickening agents with one another.

16. The oral care composition of claim 4, wherein the one or more fructans are present in an amount of from about 0.1 wt % to about 10 wt %, based on the total weight of the oral care composition.

17. The oral care composition of claim 6, wherein the polysaccharide comprises the polysaccharide gum.

18. The oral care composition of claim 17, wherein the polysaccharide gum comprises xanthan gum.

19. The oral care composition of claim 9, wherein the thickening agents are present in an amount of from about 0.05 wt % to about 2 wt %, based on the total weight of the oral care composition.

20. The oral care composition of claim 1, wherein:
the one or more fructans comprise inulin, wherein the inulin comprises a degree of polymerization of from 2 to less than 10, wherein the one or more fructans are present in an amount of from about 0.05 wt % to about 2 wt %, based on the total weight of the oral care composition;
the thickening agents comprise a polymer, wherein the polymer comprises xanthan gum;
the fructans and the thickening agents are present in a weight ratio of from about 0.5:1 to about 5:1.
